(19) United States
(12) Patent Application Publication
(10) Pub. No.: US 2004/0024036 A1
(43) Pub. Date: Feb. 5, 2004

(54) Pharmaceutical composition for transdermal delivery of befloxatone

(76) Inventors: Anne Charlier, Paris (FR); Louis Henrion, Paris (FR)

Correspondence Address: SANOFI-SYNTHELABO INC., 9 GREAT VALLEY PARKWAY, P.O. BOX 3026, MALVERN, PA, 19355, US

(21) Appl. No.: 10/343,358

(22) PCT Filed: Jul. 31, 2001
(86) PCT No.: PCT/FR01/02494

(30) Foreign Application Priority Data
Aug. 1, 2000 (FR) 0010135

(51) Int. Cl. A61K 31/421; A61K 9/70

(52) U.S. Cl. 514/376; 424/449

(57) ABSTRACT

The subject of the present invention is a pharmaceutical composition for transdermal administration of befloxatone, which may take the form of a gel, of an ointment or of an emulsion for local administration, of a transdermal patch or of a film deposited by a spray, characterized in that it comprises at least one absorption promoter.

The subject of the present invention is a pharmaceutical composition for transdermal administration of befloxatone.

More specifically, the present invention relates to a transdermal pharmaceutical composition formulated so as to allow the absorption of befloxatone through the skin at a chosen site of application.

For the purposes of the present invention, the term befloxatone is understood to mean 3-[4-(4,4,4-trifluoro-3(R)-ydroxybutoxy)phenyl5(R)-methoxymethyl-2-oxazolidinone, which is known for its antidepressant activity. It is a reversible inhibitor of MAO-A comprising both a very high affinity for the A isoform (MAO-A) and a very good selectivity toward the B isoform (MAO-B), and which does not affect noradrenalin (NA), serotonin (5-HT) or dopamine (DA) reuptake.

Its chemical synthesis is described in EP 424244.

The aim of the present invention is to obtain a controlled release of the active ingredient so as to avoid repeated daily doses of active ingredient when it is administered conventionally by the oral route. Treatment compliance is thus markedly improved.

The present invention relates to a pharmaceutical composition for transdermal administration of befloxatone, intended in particular for smoking cessation, for the treatment of depressive states or of obesity.

Another aim of the present invention is to allow rapid absorption of befloxatone at the site of application with a befloxatone level sufficient to achieve the desired therapeutic effect.

In the context of the present invention, the quantities, unless otherwise stated, are expressed in % by weight of the total composition.

More specifically, the invention consists of a pharmaceutical composition, characterized in that it comprises an active ingredient consisting of befloxatone, and at least one absorption promoter, said composition being formulated to allow transcutaneous administration.

The pharmaceutical composition may take the form of a gel, of an ointment or of an emulsion for local administration, of a transdermal patch or of a film deposited by a spray.

In the case of the gel or ointment, other excipients may also be added to the compositions according to the present invention. There may thus be mentioned conventional excipients such as perfumes, essential oils, preservatives, soothing moisturizing agents, or colorings.

The preferred mode of application is a transdermal film, in particular by means of a patch or vaporization of a spray which, upon evaporation of the solvent, leaves a film on the skin. A transdermal film allows slow and uniform administration of the active ingredient. The patient's autonomy with respect to their treatment is thus promoted. The patch makes it possible, for example, to obtain a release of the composition which can last between 8 and 72 hours.

Thus, more particularly, each composition entering into the preparation of this transdermal film comprises, in addition to the polymer, one to three absorption promoters as defined below (single, binary or ternary systems as described in the examples).

Thus, the term absorption promoter is understood to mean a pharmaceutically acceptable compound which makes it possible to improve the passage of the active ingredient across the skin. To this end, the absorption promoter can in particular modify the permeability or change the state of the surface of the skin so as to facilitate the passage.

The absorption promoters may belong to the following different categories: (i) alcohols comprising from 2 to 36 carbon atoms, esterified or otherwise with organic acids comprising from 1 to 6 carbon atoms, (ii) fatty acids comprising from 5 to 30 carbon atoms, esterified or otherwise with alcohols comprising from 1 to 6 carbon atoms, (iii) alkali and alkaline-earth metal salts of fatty acids comprising from 5 to 30 carbon atoms.

Among the alcohols, there may be particularly mentioned ethanol, isopropanol and fatty alcohols of formula R₁OH where R₁ represents a saturated or unsaturated, linear or branched alkyl radical, and comprising 6 to 30 carbon atoms. Other alcohols such as benzyl alcohol and propylene glycol may be used as absorption promoters.

As fatty acids which may be used as absorption promoters, there may be mentioned those of formula R₂COOH where R₂ represents a saturated or unsaturated, linear or branched alkyl radical, and comprising 6 to 30 carbon atoms. These fatty acids can form esters with methyl and ethyl alcohols for example, or with glycerol in the form of mono-, di- or triesters.

Caprylic, capric, lauric, stearic and oleic acids are preferred among the fatty acids which are absorption promoters.

Likewise, among the esterified fatty acids, isopropyl myristate or propylene glycol monolaurate (Lauroglycol®) will be preferred.

The alkali and alkaline-earth metal salts of fatty acids comprising from 5 to 30 carbon atoms which may be mentioned are the sodium, potassium or calcium salts of the fatty acids mentioned above.

Other absorption promoters may also be cited, among which are surfactants and dioxolanes.

According to a particular embodiment of the invention, when the transdermal film is applied with the aid of a transdermal patch, the latter may consist of a matrix system, a reservoir system or a system consisting of successive coatings. The transdermal device may in addition include constituents suitable for producing the system, for ensuring its preservation and for allowing its use.

In the case of a matrix system, these constituents may be divided into three groups: passive supports, active constituents of the system, adhesives. The passive support may be a metal, for example aluminum, film, a nonwoven fabric or a nonwoven network of natural or artificial fibers, a polymeric film such as polyethylene, polypropylene, polytetrafluoroethylene, cellulosic, acrylic or vinyl polymer, silicone, acrylonitrile, and the like. The active constituents of the system may be polymeric films or matrices such as: polyethylene, polypropylene, polytetrafluoroethylene, cellulosic, acrylic or vinyl polymer, silicone, acrylonitriles, and the like. The adhesive(s) may consist of natural or synthetic rubber, polyisobutylene, polyacrylates, polyvinyl ethers, and the like.

The transdermal devices preferred in the context of the present invention are those which comprise a polymeric matrix such as those described above and still more particularly those for which the polymer constituting the matrix system is also adhesive, like polyacrylates. The composition in the case of a transdermal patch comprising a polymeric matrix is characterized in that it comprises (i) befloxatone, (ii) one to three absorption promoters and (ii) a polymer.

According to the present invention, it is possible to obtain transdermal patches according to methods which are conventional for persons skilled in the art.

According to another particular embodiment of the invention, when the transdermal film is applied with the aid of a spray, the latter may be provided in the form of a solution. It is characterized in that it comprises, in addition (i) befloxatone, (ii) one to three absorption promoters, (iii) a polymer or copolymer forming a flexible film after evaporation of the solvent and (iv) a solvent capable of dissolving the constituents (i), (ii) and (iii) above and which can be easily evaporated.

The composition is prepared conventionally by mixing the constituents.

Among the polymers or copolymers capable of forming a flexible film after evaporation, there are chosen more particularly cellulosic polymers or copolymers, in particular because they exhibit, after drying, appropriate resistance to abrasion and appropriate mechanical stability. For this reason, cellulosic matrices of this type can be rinsed with water without fear of deterioration or of elimination of the active ingredients.

By way of example of such cellulosic polymers or copolymers which can be used in the spray solutions of the invention, there may be mentioned ethyl cellulose, cellulose acetate butyrate, cellulose acetate propionate or a hydroxypropylmethylcellulose grafted or otherwise such as hydroxypropylmethylcellulose acetate succinate.

Ethyl cellulose represents the preferred cellulosic polymer and, consequently, the polymeric matrix of choice for the formation of a flexible film in contact with the skin.

In addition, the polymeric matrix may consist of a vinylpyrrolidone/vinyl acetate such as polyvinylpyrrolidone/vinyl acetate copolymer.

Most of the promoters of absorption of an active ingredient being generally a solvent thereof, the term solvent is here, unless otherwise stated, reserved for the polymer solvent, in particular in the present case where the formulation is a spray film. Said solvent is then in this case both the solvent for the polymer and for the active ingredient.

Thus, the solvents may be organic solvents such as ethanol, isopropanol, ethyl acetate, acetone, diethyl ether, or alternatively a mixture thereof. Among these volatile solvents, ethanol which is particularly physiologically acceptable, may be preferably used in a formulation for a spray film.

A pharmaceutical composition according to the invention, regardless of the form which it takes, in particular a transdermal patch or a spray solution, may comprise from 1 to 20% of befloxatone.

The content of absorption promoter relative to the total quantity of active ingredient may vary from 5 to 40%.

The content of solvent in the spray solution may vary in particular according to the nature and the content of polymer, where appropriate, but also according to the nature and content of absorption promoter. Based on these various parameters, persons skilled in the art will be able to determine the required solvent content.

### EXAMPLE 1

### Flow measurements Procedure

An ex vivo model using human skin collected during plastic surgery operations (breast and abdominal plastic surgery) and carefully preserved in order to ensure that its physiological properties are maintained, was used to test the compositions and their efficacy in improving transdermal passage. This model was initially described by T. J. Franz (Current Problems in Dermatology, 7,58-68, 1978) and by H. Durrheim et al. (Journal of Pharmaceutical Sciences, 69,7,781-786, 1980).

The stratum corneum, the epidermis and part of the dermis are excised with the aid of a dermatome at a thickness of 250 μm. The study of transdermal passage is carried out in diffusion cells. These cells comprise two compartments (donor and receiving compartments). The receiving compartment is subjected to magnetic stirring at 600 rpm. The surface area for exchange between the two compartments is 0.636 cm². The whole is maintained at 32° C. (temperature of the skin surface in humans) by circulating thermostatted water around the compartments.

Pieces of dermatomed skin, cut into cubes of size 1.5 to 2 cm are stretched between the two compartments of the diffusion cell. The stratum corneum face is placed on the donor side and the epidermal face on the receiving side.

The donor compartment contains the saturated befloxatone solution at 32° C. to be tested. The receiving compartment contains a 9% NaCl solution (physiological saline).

The experiment is carried out for 24 to 48 hours. During the first few hours, the samples are collected in close succession in order to determine the diffusion lag time.

The collected samples, stored at 32° C., are assayed by HPLC.

The transcutaneous flow at equilibrium is determined by the slope of the linear section of the diffusion curve corresponding to the quantity of befloxatone which has diffused as a function of time.

Results

The results are assembled in tables 1 to 4.

They relate to the measurement of the passage of befloxatone solubilized in mixtures of promoters and/or solvents which can enter either into the patch composition, or into the spray composition.

In the “lag-time” column is the time separating the start of the measurement and the moment when the diffusion becomes really linear.

In the “flow” column is the slope calculated for the linear section of the diffusion; it is the transcutaneous flow.

In the “concentration” column is the initial befloxatone concentration in the donor medium.

1. FLOW OBTAINED IN SOLUTION IN SINGLE SYSTEMS

| | | Flow | |
|---|---|---|---|
| | | (μg/cm²/h) | Concentration |
| Composition | Lag time (h) | c.v. (%) | (mg/mL) |
| Propylene glycol | −9.1 ± 16.3 | 2.41 ± 1.69 | 57.4-60.3 |
| | | 70% n = 4 | |
| Isopropyl | 2.4 ± 0.7 | 3.57 ± 1.09 | 2.8-3.1 |
| myristate | | 31% n = 3 | |
| Ethoxylated | 5.3 ± 9.2 | 7.64 ± 7.29 | 36.6-43.5 |
| C₈-C₁₀ glyceride | | 95% n = 4 | |
| Labrafac ®, | | | |
| hydrophilic | | | |
| (Gattefosse) | | | |
| Propylene glycol | 2.0 ± 1.1 | 19.82 ± 2.63 | 23.2-25.7 |
| monolaurate | | 13% n = 4 | |
| Lauroglycol ® | | | |
| (Gattefosse) | | | |

2. FLOW OBTAINED IN SOLUTION IN BINARY SYSTEMS

| | | Flow | |
|---|---|---|---|
| | | (μg/cm²/h) | Concentration |
| Composition | Lag time (h) | c.v. (%) | (mg/mL) |
| Isopropyl | 10.6 ± 5.9 | 6.02 ± 0.41 | 3.2 |
| myristate/ | | 7% n = 2 | |
| caprylic acid | | | |
| Lauroglycol ®/ | 2.3 ± 1.2 | 18.91 ± 1.66 | 19.7-23.3 |
| oleic acid 90/10 | | 9% n = 3 | |
| Lauroglycol ®/ | 2.9 ± 0.9 | 19.39 ± 4.87 | 20.4-24.3 |
| linoleic acid | | 25% n = 4 | |
| 90/10 | | | |
| Lauroglycol ®/ | 4.6 ± 0.2 | 21.14 ± 4.28 | 21.4-21.7 |
| caprylic acid | | 18% n = 3 | |
| 90/10 | | | |

3. FLOW OBTAINED IN SOLUTION IN TERNARY SYSTEMS

| | | Flow | |
|---|---|---|---|
| | | (μg/cm²/h) | Concentration |
| Composition | Lag time (h) | c.v. (%) | (mg/mL) |
| Lauroglycol ®/ | 6.2 ± 2.2 | 17.19 ± 4.67 | 16.0-17.0 |
| ipm/caprylic | | 27% n = 4 | |
| acid | | | |
| 80/10/10 | | | |
| Lauroglycol ®/pg/ | 4.2 ± 1.2 | 39.41 ± 8.80 | 25.1-28.0 |
| caprylic acid | | 22% n = 4 | |
| 80/10/10 | | | |
| Lauroglycol ®/pg/ | 13.4 ± 4.4 | 40.35 ± 14.69 | 49.6-52.5 |
| caprylic acid | | 36% n = 4 | |
| 45/45/10 | | | |

4. FLOW OBTAINED IN SOLUTION
IN THE PRESENCE OF ETHANOL

| | | Flow | |
|---|---|---|---|
| | | (μg/cm²/h) | Concentration |
| Composition | Lag time (h) | c.v. (%) | (mg/mL) |
| Ethanol | 10.5 ± 1.3 | 5.0 ± 3.5 | 209.2-271.3 |
| | | 70% n = 4 | |
| Ethanol/caprylic | 0.2 ± 2.6 | 2.78 ± 2.25 | 216.2 |
| acid | | 81% n = 3 | |
| 90/10 | | | |
| Ethanol/oleic | 11.5 ± 1.7 | 4.5 ± 3.0 | 196.8 |
| acid | | 67% n = 2 | |
| 90/10 | | | |
| Lauroglycol ®/ | 6.3 ± 6.7 | 14.17 ± 3.16 | 137.9 |
| ethanol | | 22% n = 3 | |
| 50/50 | | | |
| Lauroglycol ®/ | 2.9 ± 0.4 | 42.03 ± 5.29 | — |
| ethanol | | 13% n = 3 | |
| 90/10 | | | |
| Lauroglycol ®/ | 14.3 ± 2.8 | 9.76 ± 9.87 | 102.6-107.1 |
| ethanol/caprylic | | 101% n = 4 | |
| acid | | | |
| 45/45/10 | | | |
| Lauroglycol ®/ | 13.4 ± 6.9 | 47.29 ± 12.20 | 29.2-37.5 |
| ethanol/caprylic | | 26% n = 4 | |
| acid | | | |
| 80/10/10 | | | |

### EXAMPLE 2

### Formulation of a Patch

An 80:10:10 weight mixture of the promoters Lauroglycol®, propylene glycol and caprylic acid is prepared. 15% of the promoter mixture is introduced into noncrosslinked Duro-Tak® 387-2353, a self-adhesive polymer which belongs to the polyacrylate family. Befloxatone is added in a proportion such that the active matrix film obtained is at a dose of 15 mg/25 cm². A Scotchpak® 1009 (a support consisting of aluminum foil coated with polyethylene serving as external support—often transparent or flesh-colored) is coated at the rate of 6 cm/s on a vacuum table with a filmograph set at 500 μm. The polymer is crosslinked by evaporating the solvents in ventilated oven for 10 min at 85° C. The Scotchpak® 1022 (3M) release (=plastic sheet coated with silicone whihc is detached and which is thrown away before application) is applied to the adhesive face of the polymer film. The film obtained is cut into patches of diameter 2.5 cm (that is 4.909 cm²) for the tests in Franz cells and into patches of diameter 3.5 cm (that is 9.621 cm²) of the tests in dissolutest. The patches obtained are weighed. The coated matrix mass per patch is calculated by subtracting from the preceding weighing the masses of the backing and of the release (that is 18 mg/cm²). The coated matrix mass expressed relative to 25 cm² is calculated. The dosage of the patches is calculated by multiplying the coated matrix mass by the percentage of active ingredients in the formulation.

The patches correspond to the following formula:

| Constituents in % of the total mass | |
|---|---|
| Lauroglycol ® (Gattefosse) | 11.5 |
| Propylene glycol (Sigma) | 1.4 |
| Caprylic acid (Sigma) | 1.4 |
| Duro-Tak ® 387-2353 (N^{al} Starch & Chemical | 81.0 |
| Befloxatone | 4.7 |
| Results of the control of the patches (expressed relative to 25 cm²) | |
| Measurements | |
| Coated matrix mass (mg) | 334.3 |
| Content of befloxatone in the | 4.7 |
| preparation (%) | |
| Real dosage (mg) | 15.71 |
| Coated thickness (μm) | 500 |
| Dry thickness (μm | 115 |

The various terms used above are defined as follows:

Coated matrix mass: mass calculated by subtracting from the weight of one cm² of crosslinked patch the weight corresponding to one cm² of backing and of one cm² of release (that is 18 mg/cm² in total), and expressed relative to the surface area of the patch (25 cm²).

Real dosage: product of the content of befloxatone in the initial formulation mixture by the coated matrix mass, and expressed relative to the surface area of the patch.

Coated thickness: matrix coating thickness before crosslinking, given by the height of the adjustment of the filmograph.

Dry thickness: matrix coating thickness after evaporation of the solvents, measured with a dial thickness gage.

1. Pharmaceutical composition for transdermal administration of befloxatone.

2. Pharmaceutical composition according to claim 1, characterized in that it comprises befloxatone and at least one absorption promoter.

3. Pharmaceutical composition according to claim 2, characterized in that the absorption promoters may be chosen from (i) alcohols comprising from 2 to 36 carbon atoms, esterified or otherwise with organic acids comprising from 1 to 6 carbon atoms, (ii) fatty acids comprising from 5 to 30 carbon atoms, esterified or otherwise with alcohols comprising from 1 to 6 carbon atoms, (iii) alkali and alkaline-earth metal salts of fatty acids comprising from 5 to 30 carbon atoms or alternatively a mixture of these absorption promoters.

4. Pharmaceutical composition according to claim 3, characterized in that the absorption promoter comprises an alcohol chosen from isopropanol, fatty alcohols of formula R₁OH, where R₁ represents a saturated or unsaturated, linear or branched alkyl radical comprising 6 to 30 carbon atoms, benzyl alcohol or propylene glycol.

5. Pharmaceutical composition according to claim 3 or 4, characterized in that the absorption promoter comprises a fatty acid chosen from those of formula R₂COOH, where R₂ represents a saturated or unsaturated, linear or branched alkyl radical comprising 6 to 30 carbon atoms, it being possible for these fatty acids to form esters with methyl and ethyl alcohols or with glycerol in the form of mono-, di- or triesters.

6. Pharmaceutical composition according to any one of claims 3 to 5, characterized in that the absorption promoter comprises an alkali or alkaline-earth metal salt of fatty acid comprising from 5 to 30 carbon atoms chosen from the sodium, potassium or calcium salts of these fatty acids.

7. Pharmaceutical composition according to any one of claims 1 to 6, characterized in that it may take the form of a gel, of an ointment or of an emulsion for local administration, of a transdermal patch or of a film deposited by a spray.

8. Pharmaceutical composition according to any one of claims 1 to 7, characterized in that it takes the form of a transdermal film.

9. Pharmaceutical composition according to claim 8, characterized in that it takes the form of a transdermel patch or of a spray solution.

10. Pharmaceutical composition in the form of a transdermal patch according to claim 9, characterized in that the latter comprises a passive support chosen from a metal film, a nonwoven fabric or a nonwoven network of natural or artificial fibers or a polymeric film or an active constituent of the system chosen from polymeric films or polymeric matrices or an adhesive chosen from natural or synthetic rubber, polyisobutylene, polyacrylates or polyvinyl ethers.

11. Pharmaceutical composition in the form of a transdermal patch according to claim 10, characterized in that the transdermal patch comprises a polymeric film chosen from polyethylene, polypropylene, polytetrafluoroethylene, a cellulosic, acrylic or vinyl polymer, silicone or alternatively acrylonitriles.

12. Pharmaceutical composition in the form of a spray solution according to claim 9, characterized in that the latter consists of a solution comprising in addition (i) befloxatone, (ii) one to three absorption promoters, (iii) a polymer or copolymer forming a flexible film after evaporation of the solvent and (iv) a solvent.

13. Pharmaceutical composition in the form of a spray solution according to claim 12, characterized in that the polymer or copolymer is a polymer or copolymer which may be chosen from ethyl cellulose, cellulose acetate butyrate, cellulose acetate propionate or a hydroxypropylmethylcellulose grafted or otherwise, or alternatively a vinylpyrrolidone/vinyl acetate copolymer.

14. Pharmaceutical composition in the form of a spray solution according to claim 12 or 13, characterized in that the solvent is chosen from ethanol, isopropanol, ethyl acetate, acetone, diethyl ether, or alternatively a mixture thereof.

15. Pharmaceutical composition according to any one of claims 1 to 14, characterized in that it comprises from 1 to 20% of befloxatone.

16. Pharmaceutical composition according to any one of claims 1 to 15, characterized in that the total content of absorption promoter relative to the total quantity of befloxatone varies from 5 to 40%.
